(19) United States
(12) Patent Application Publication
(10) Pub. No.: US 2002/0160042 A1
(43) Pub. Date: Oct. 31, 2002

(54) Injection molding method for neutral and acidic-group containing (meth)acrylate copolymers

(76) Inventors: Hans-Urich Petereit, Darmstadt (DE); Thomas Beckert, Darmstadt (DE); Manfred Assmus, Bickenbach (DE); Werner Hoess, Heusenstamm (DE); Wolfgang Fuchs, Alsbach (DE); Hartmut Schikowsky, Darmstadt (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(21) Appl. No.: 09/913,720

(22) PCT Filed: Dec. 9, 2000
(86) PCT No.: PCT/EP00/12467

(30) Foreign Application Priority Data
Dec. 17, 1999 (DE) 19961334.6

(51) Int. Cl. A61K 9/20; C08J 3/20; C08L 33/06; B29C 45/00

(52) U.S. Cl. 424/464; 264/102; 264/328.16; 264/328.18; 524/560

(57) ABSTRACT

The invention relates to a process for producing mouldings by injection moulding, the steps in the process being

A) Melting a mixture made from

a) a (meth)acrylate copolymer composed of from 40 to 100% by weight of free-radical-polymerized C₁-C₄-alkyl esters of acrylic or methacrylic acid and from 0 to 60% by weight of (meth)acrylate monomers having an anionic group in the alkyl radical, where the copolymer comprises

b) from 0.1 to 3% by weight of a release agent, and, where appropriate, the mixture may comprise

c) from 0 to 50% by weight of a drier,

d) from 0 to 30% by weight of a plasticizer,

e) from 0 to 100% by weight of additives or auxiliaries,

f) from 0 to 100% by weight of an active pharmaceutical ingredient,

g) from 0 to 20% by weight of another polymer or copolymer,

where the amounts given for components b) to g) are based on the (meth)acrylate copolymer a) and

the mixture prior to melting has a content of more than 0.5% by weight of low-boiling constituents with vapour pressure of at least 1.9 bar at 120° C.,

B) Devolatilizing the mixture in the thermoplastic state at temperatures of at least 120° C., thereby lowering to not more than 0.5% by weight the content of the low-boiling constituents with vapour pressure of at least 1.9 bar at 120° C.,

C) Injecting the molten and devolatilized mixture into the mould cavity of an injection mould, the temperature of the mould cavity being below the glass transition temperature of the (meth)acrylate copolymer by at least 10° C., cooling the molten mixture, and removing the resultant moulding from the mould.

### PRIOR ART

U.S. Pat. No. 5,644,011 relates to coating compositions and binders for drug forms comprising copolymers of 10 to 25% by weight of methacrylic acid, 40 to 60% by weight of methyl acrylate and 20 to 40% by weight of methyl methacrylate. Application takes place from aqueous dispersion or organic solution.

EP 0 704 207 A2 describes thermoplastics for encapsulating drugs which are soluble in intestinal fluid. These are copolymers made from 16 to 40% by weight of acrylic or methacrylic acid, from 30 to 80% by weight of methyl acrylate and from 0 to 40% by weight of other alkyl esters of acrylic acid and/or methacrylic acid.

In the example, copolymers of this type are melted at 160° C. and mixed after addition of 6% by weight of glycerol monostearate. The mixture is crushed and ground to give a powder. The powder is charged to the antechamber of a transfer mould and is injected into the mould cavity at 170° C. under a pressure of 150 bar through an opening of width 0.5 mm. Cooling gives thin-walled drug capsules which are bubble-free and slightly opaque. There is no disclosure of particular measures for removing low-boiling constituents immediately prior to the injection moulding process.

### OBJECT AND MANNER OF ACHIEVING THIS ABJECT

The object was to provide a process more advanced than that of EP 0 704 207 A2 and permitting neutral or anionic (meth)acrylate copolymers to be injection moulded in such a way as to minimize contamination of the plant and at the same time allow high throughputs to be obtained of mouldings free from fracture and streaking, with only a low level of rejects. The mouldings obtained should meet high mechanical requirements and therefore be suitable for carrying or containing active pharmaceutical ingredients, e.g. as capsules (hard capsules) or parts.

The object is achieved by way of a process for producing mouldings by injection moulding, the steps in the process being

A) Melting a Mixture Made From

a) a (meth)acrylate copolymer composed of from 60 to 100% by weight of free-radical-polymerized C₁-C₄-alkyl esters of acrylic or methacrylic acid and from 0 to 50% by weight of (meth)acrylate monomers having an anionic group in the alkyl radical, where the copolymer comprises

b) from 0.1 to 3% by weight of a release agent, and, where appropriate, the mixture may comprise

c) from 0 to 50% by weight of a drier,

d) from 0 to 30% by weight of a plasticizer,

e) from 0 to 100% by weight of additives or auxiliaries,

f) from 0 to 100% by weight of an active pharmaceutical ingredient,

g) from 0 to 20% by weight of another polymer or copolymer,

where the amounts given for components b) to g) are based on the (meth)acrylate copolymer a) and

the mixture prior to melting has a content of more than 0.5% by weight of low-boiling constituents with vapour pressure of at least 1.9 bar at 120° C.,

B) Devolatilizing the mixture in the thermoplastic state at temperatures of at least 120° C., thereby lowering to not more than 0.5% by weight the content of the low-boiling constituents with vapour pressure of at least 1.9 bar at 120° C.

C) Injecting the molten and devolatilized mixture into the mould cavity of an injection mould, the temperature of the mould cavity being below the glass transition temperature of the (meth)acrylate copolymer by at least 10° C., cooling the molten mixture, and removing the resultant moulding from the mould.

By means of the process of the invention it is possible to obtain novel injection mouldings which meet the requirements for high mechanical strength and high heat resistance.

### WORKING OF THE INVENTION

The process of the invention for producing mouldings by means of injection, moulding divides into steps A), B) and C).

Step A) is the melting of a mixture made from

a) a (meth)acrylate copolymer composed of from 45 to 100% by weight of free-radical-polymerized C₁-C₄-alkyl esters of acrylic or methacrylic acid and from 0 to 55% by weight of (meth)acrylate monomers having an anionic group in the alkyl radical, where the copolymer comprises

b) from 0.1 to 3% by weight of a release agent, and, where appropriate, the mixture may comprise

c) from 0 to 50% by weight of a drier,

d) from 0 to 30% by weight of a plasticizer,

e) from 0 to 100% by weight of additives or auxiliaries,

f) from 0 to 100% by weight of an active pharmaceutical ingredient,

g) from 0 to 20% by weight of another polymer or copolymer,

where the amounts given for components b) to g) are based on the (meth)acrylate copolymer a) and

the mixture prior to melting has a content of more than 0.5% by weight of low-boiling constituents with vapour pressure of at least 1.9 bar at 120° C.

The melting of the copolymer, which is in the form of pallets or powder, preferably takes place in an extruder at a temperature of from 120 to 250° C.

### THE MIXTURE

The mixture is composed of components a) and b), and also optionally c) to g).

### The (Meth)Acrylate Copolymer a)

The (meth)acrylate copolymer is composed of from 40 to 100% by weight, preferably from 45 to 99% by weight, in particular from 85 to 95% by weight, of free-radical-polymerized C₁-C₄-alkyl esters of acrylic or of methacrylic acid, and may comprise from 0 to 60% by weight, preferably from 1 to 55% by weight, in particular from 5 to 15% by weight, of (meth)acrylate monomers having an anionic group in the alkyl radical.

In particular C₁-C₄-alkyl esters of acrylic or methacrylic acid are methyl methacrylate, ethyl methacrylate, butyl methacrylate, methyl acrylate, ethyl acrylate and butyl acrylate.

A (meth)acrylate monomer having an anionic group in the alkyl radical may be acrylic acid, for example, but is preferably methacrylic acid

Examples of suitable (meth)acrylate copolymers are neutral copolymers made from 20 to 40% by weight of ethyl acrylate and from 60 to 80% by weight of methyl methacrylate (EUDRAGIT® NE grade).

Anionic (meth)acrylate copolymers made from 40 to 60% by weight of methacrylic acid and from 60 to 40% by weight of methyl methacrylate, or from 60 to 40% by weight of ethyl acrylate (EUDRAGIT® L or EUDRAGIT® L100-55 grades) are also suitable.

Anionic (meth)acrylate copolymers made from 20 to 40% by weight of methacrylic acid and from 80 to 60% by weight of methyl methacrylate (EUDRAGIT® S grade) are also suitable.

(Meth)acrylate copolymers composed of from 10 to 30% by weight of methyl methacrylate, from 50 to 70% by weight of methyl acrylate and from 5 to 15% by weight of methacryllc acid (EUDRAGIT® FS grade) are particularly highly suitable.

The copolymers are obtained in a manner known per se by free-radical bulk, solution, bead or emulsion polymerization. Prior to processing, they must be brought within the particle size range of the invention by suitable grinding, drying or spraying processes. Simple crushing of extruded and cooled pelletized extrudates, or die-face cutting, may be used for this purpose.

The use of powders can be advantageous, in particular during mixing with other powder or liquids. Suitable equipment for producing the powders, e.g. air-jet mills, pinned-disc mills, compartmentalized mills, is familiar to the person skilled in the art. Appropriate screening steps may be included, where appropriate. An example of a mill suitable for large-scale industrial quantities is a counterflow mill (Multi No. 4200), operated at about 6 bar gauge pressure.

### Release Agent (Mould-Release Agent) b)

The mixture comprises from 0.1 to 3% by weight, preferably from 0.2 to 1% by weight, of a release agent, based on the (meth)acrylate copolymer.

Unlike driers, mould-release agents have the property of reducing the strength of adhesion between the mouldings and the surface of the mould in which the moulding is produced. This permits a production of mouldings which have not suffered breakage or geometric deformation. Mould-release agents are mostly incompatible, or partially compatible, with the polymers in which they are particularly effective. The incompatibility or partial compatibility causes migration into the transitional interface between mould wall and moulding, when the melt is injected into the mould cavity. The melting point of the mould-release agent has to be below the processing temperature of the polymer by from 20 to 100° C. in order to permit particularly advantageous migration of this agent.

Examples of release agents (mould-release agents) are:

esters of fatty acids or fatty amides, aliphatic long-chain carboxylic acids, fatty alcohols and esters of these, montan waxes, paraffin waxes, and metal soaps, and particular mention should be made of glycerol monostearate, stearyl alcohol, glycerol behenate, cetyl alcohol, palmitic acid, canauba wax, beeswax, etc.

### Drier c)

The mixture may comprise from 0 to 50% by weight, preferably from 10 to 30% by weight, of a drier, based on the (meth)acrylate copolymer.

Driers have the following properties: they have large specific surface areas, are chemically inert, are free-flowing, and consist of fine particles. These properties mean that they become advantageously and uniformly distributed in melts and reduce the tack of polymers in which highly polar comonomers are present as functional groups.

Examples of driers are:

aluminium oxide, magnesium oxide, kaolin, talc, silica (Aerosils), barium sulphate, carbon black and cellulose.

### Plasticizer d)

The mixture may comprise from 0 to 30% by weight, preferably from 0.5 to 15% by weight, of a plasticizer, based on the (meth)acrylate copolymer.

The addition of plasticizer reduces the brittleness of the mouldings. The result is a reduction in the proportion of broken mouldings after demoulding. Without plasticizer, the proportion of mouldings satisfactorily removed from the mould is about 85% for most mixtures. With addition of plasticizer, the proportion of breakage on demoulding can be reduced, mostly resulting in a rise in yields to 95-100%.

Substances suitable as plasticizers generally have a molecular weight of from 100 to 20 000 and contain one or more hydrophilic groups in the molecule, e.g. hydroxyl groups, ester groups or amino groups. Suitable substances are citrates, phthalates, sebacates, castor oil. Examples of suitable plasticizers are alkyl citrates, glycerol esters, alkyl phthalates, alkyl sebacates, sucrose esters, sorbitan esters, dibutyl sebacate and polyethylene glycols 400 g/mol to 20 000 g/mol. Preferred plasticizers are tributyl citrate, triethyl citrate, acetyltriethyl citrate, dibutyl sebacate and diethyl sebacate.

### Additives or Auxiliaries e)

The mixture may comprise from 0 to 100% by weight of conventional pharmaceutical additives or auxiliaries, based on the (meth)acrylate copolymer.

Examples which should be mentioned here are stabilizers, dyes, antioxidants, wetting agents, pigments, lustre agents, etc.

### Active Pharmaceutical Ingredient (f)

The mixture may comprise from 0 to 100% by weight of one or more active pharmaceutical ingredients, based on the (meth)acrylate copolymer. The active pharmaceutical ingredients used here are those which do not decompose at the processing temperature.

The drugs (active pharmaceutical ingredients) used for the purposes of the invention are those intended for use in the bodies of humans or of animals, in order to

1. cure, alleviate, prevent or detect diseases, suffering, bodily injury or pathological symptoms,

2. permit detection of the condition, the state, or the functions of the body, or of mental states,

3. replace body fluids or active materials produced by the human body or by the bodies of animals,

4. defend against, eliminate, or render harmless pathogens, parasites or exogenous substances, or

5. influence the condition, the state, or the functions of the body, or influence mental states.

Reference works, such as the Roten Liste or the Merck Index, should be referred to for commonly used drugs.

According to the invention use may be made of any active ingredient which complies with the desired therapeutic action in the sense of the definition above and which has sufficient stability or ability to penetrate the skin.

Without any claim to completeness, the following are important examples (classes and individual substances);

analgesics,

antiallergics, antiarrhythmics,

antibiotics, chemotherapeutics, antidiabetics, antidotes,

antiepileptics, antihypertensives, antihypotensives,

anticoagulants, antimycotics, anti-inflammatories,

beta-receptor blockers, calcium antagonists and ACE inhibitors,

broncholytics/antiasthmatics, cholinergics, corticoids (Interna),

dermatics, diuretics, enzyme inhibitors, enzyme preparations and transport proteins,

expectorants, geriatrics, gout remedies, influenza remedies,

hormones and their inhibitors, hypnotics/sedatives, cardiac stimulants, lipid-lowering agents,

parathyroid hormones/calcium metabolism regulators,

psychopharmaceuticals, sex hormones and their inhibitors,

spasmolytics, sympatholytics, sympathomimetics, vitamins, wound treatment agents, cytostatics.

Examples of suitable active ingredients for inserting into the mouldings (capsules) or else for incorporation into the mouldings are: ranitidine, simvastatin, enalapril, fluoxetine, amlodipine, amoxicillin, sertralin, nifidipine, ciprofloxacin, acyclovir, lovastatin, epoetin, paroxetine, captopril, nabumetone, granisetron, cimetidine, ticarcillin, triamterene, hydrochlorothiazide, varapamil, paracetamol, morphine derivatives, topotecan or the salts used pharmaceutically.

### Other Polymers or Copolymers g)

The mixture may comprise from 0 to 20% by weight of another polymer or copolymer, based on the (meth)acrylate copolymer.

To control active ingredient release, in certain cases it can be advantageous to admix other polymers The proportion of other polymers in the mixture, however, is not more than 20% by weight, preferably not more than 10% by weight, in particular from 0 to 5% by weight, based on the (meth)acrylate copolymer.

Examples of these other polymers are: polyvinylpyrrolidones, polyvinyl alcohols, cationic (meth)acrylate copolymers made from methyl methacrylate and/or ethyl acrylate and 2-dimethylaminoethyl methacrylate (EUDRAGIT® E100), carboxymethylcellulose salts, hydroxypropylcellulose (HPMC), neutral (meth)acrylate copolymers made from methyl methacrylate and ethyl acrylate (dry matter from EUDRAGIT® NE 30 D), copolymers made from methyl methacrylate and butyl methacrylate (PLASTOID® B) or (meth)acrylate copolymers with quaternary ammonium groups and containing trimethylammoniumethyl methacrylate chloride as monomer (EUDRAGIT® RL and/or EUDRAGIT® RS).

### Low-Boiling Constituents

The commercially available form of the (meth)acrylate copolymer known per se almost always has a content greater than 0.5% by weight of low-boiling constituents With a vapour pressure of at least 1.9 bar at 120° C.

The content of these constituents is usually in the range from 0.7 to 2.0% by weight. The low-boiling constituents are mainly water absorbed from the moisture present in air or derived from the polymer preparation process.

### Step B) of the Process

Devolatilization of the mixture at temperatures of at least 120° C., preferably at least 150 C. and not more than 250° C., lowering the content of the low-boiling constituents with a vapour pressure of at least 1.9 bar at 120° C. to not more than 0.5% by weight, preferably not more than 0.2% by weight, particularly preferably not more than 0.1% by weight. This can prevent undesired sudden evolution of gas during the injection moulding procedure in step c) of the process, resulting in bubble formation or foaming within the resultant moulding, which would then be unusable.

Since the stated (meth)acrylate copolymers either have a low glass transition temperature, and thus may become adhesive even at low temperatures, or else are thermally unstable, low-boiling constituents cannot generally be removed by simple drying at elevated temperature.

The devolatilization step b) is therefore carried out, preferably by extrusion drying by means of an extruder with a devolatilizing section, or by means of an injection moulding system which has an injection mould preceded by a vent.

For more efficient devolatilization it is also possible to install a vacuum-generating pump (e.g. water pump) at the vent of the extruder or of the injection moulding machine. The reduced pressures which can be generated thereby lead to more substantial removal of the low-boiling constituents, such as moisture, from the melt. Reduced pressures which can be generated thereby may be from 800 mbar to 10 mbar.

Without any other steps for removing low-boiling constituents, the devolatilized extrudate obtained by extrusion drying in an extruder with a devolatilizing section can be charged immediately to the injection moulding machine and processed directly to give mouldings.

In the case of devolatilization in an injection moulding system comprising a vent in the injection moulding cylinder, the devolatilization takes place prior to injection of the polymer melt into the injection mould, by means of the vent mentioned in the injection moulding cylinder.

### Step C) of the Process

Injection of the molten and devolatilized mixture into the mould cavity of an injection mould, the temperature of the mould cavity being below the glass transition temperature of the (meth)acrylate copolymer by at least 10° C., preferably at least 12° C., particularly preferably at least 15° C., in particular at least 25° C., or even at least 35° C., cooling the molten mixture, and removing the resultant moulding from the mould.

The thermoplastic processing takes place in a manner known per se by means of an injection moulding machine at temperatures in the range from 80 to 220° C., in particular from 120 to 160° C., and at pressures of from 60 to 400 bar.

If the glass transition temperature of the (meth)acrylate copolymer used is in the range of from 40° C. to 80° C., for example, the mould temperature is correspondingly lower, e.g. not more than 30° C. or not more than 20° C., so that only a short time passes after the injection procedure before the copolymer solidifies in the mould and the finished moulding can be removed or demoulded.

The mouldings can be removed from the mould cavity of the injection mould without breakage and have a uniform, compact and defect-free surface. The moulding has mechanical strength and, respectively, flexibility and fracture resistance.

In particular, it has impact strength to ISO 179 of at least 15 kJ/m², preferably at least 18 kJ/m², particularly preferably at least 20 kJ/m², measured on test specimens.

The heat distortion temperature VST (A10) is from about 30° C. to 60° C., measured on test specimens to ISO 306.

Examples of the shape of the mouldings obtained according to the invention are that of a capsule, of part of a capsule, e.g. of half of a capsule, or of a hard capsule used to contain an active pharmaceutical ingredient. Active ingredients can be inserted, e.g. in the form of pellets, and the two parts of the capsule are then joined by adhesive bonding, welding by laser, ultrasound or microwaves, or by means of a snap connection.

According to the invention, this process may also be used to combine with one another capsules made from differing materials (e.g. gelatin, partially hydrolysed starch, HPMC or non-identical methacrylates). The moulding may therefore also be a part of a dosage unit.

Other shapes are also possible, for example tablet shapes or lenticular shapes. In this case the active pharmaceutical ingredient is present in the composition before it is used for injection moulding. When the product has reached its final shape, the distribution of the active ingredient is very uniform, in crystalline form (solid dispersion) or in dissolved form) (solid solution).

### EXAMPLES

### Example 1

### Moulding Soluble in Intestinal Fluid

10 kg of a (meth)acrylate copolymer in pellet form, composed of methyl methacrylate, methyl acrylate and methacrylic acid in a ratio of 25:65:10, are placed in a 30 I stainless steel mixing vessel, and 12.5 g of stearyl alcohol (0.25% by weight) are weighed in, and the mixture is then mixed on a tumbling mixer for 5 min. The resultant mixture was charged to a Leistritz LMS 30.34 twin-screw extruder in order to prepare a composition of the invention. The melt temperature set was 180° C., with a screw rotation rate of 120 rpm.

At a position 50% along the total length of the twin-screw extruder the barrel wall has an opening via which 1% of triethyl citrate, based on the amount of polymer, is pumped in by means of a membrane pump. Downstream of a mixing zone for homogenizing the mixture, the barrel has a vent with an opening into the surroundings. Steam can be observed to emerge from the vent. A die is used to shape 4 extrudates from the extruder, and these are drawn off by way of a cooled metal plate and chopped to give pellets. The moisture content of the resultant pellets was determined as 0.08% by the Karl Fischer method. The water content found on testing the in-going pellets before extrusion was 1.2%.

### Injection Moulding of the Resultant Pellets

The resultant devolatilized and pelletized mixture was charged to the hopper of an injection moulding machine (Arburg Allrounder 250-125) and injection moulded to give capsules.

A 4-fold injection mould with a cold-runner feed system was used. The length of the capsules is 16 mm and their central external diameter is 6.8 mm, narrowing to 4 mm at the closed end, and their wall thickness is 0.6 mm.

The following temperatures were set on the injection moulding machine: zone 1 (feed zone): 70° C., zone 2: 160° C., zone 3; 160° C., zone 4: 160° C., zone 5 (die): 130° C.

Injection pressure: 60 bar, hold pressure: 50 bar, back pressure: 3 bar mould temperature: 17° C.

After injection of the melt and a hold pressure time of 6 s, followed by a cooling time of 18 s, the mould was opened and the capsules demoulded. The mouldings could be removed from the mould without breakage. The capsules obtained were transparent and mechanically stable and could be utilized for further testing.

After 300 shots had been injection moulded, the cycle was interrupted in order to assess the surface of the mould. No deposit could be seen. The polished mould surface is shiny and metallic, with high gloss.

### Example 2

### Comparative Example

A mixture was prepared in accordance with the example of EP 0 704 207 A2. Instead of the copolymers described in that text, use was made of 10 kg of a (meth)acrylate copolymer in pellet form, composed of methyl methacrylate, methyl acrylate and methacrylic acid in a ratio of 25:65:10, and this was mixed with 6% by weight of glycerol monostearate in accordance with EP 0 704 207 A2.

To this end, 10 kg of the (meth)acrylate copolymer and 600 g of glycerol monostearate were continuously metered into the feed zone of the twin-screw extruder via gravimetric metering equipment.

The components were incorporated uniformly into the melt in the extruder using a screw rotation rate of 120 rpm and a melt temperature of 160° C.

As in Example 1, the pellets were charged to the Injection moulding machine and processed while retaining the setting of the parameters.

After 14 injection moulding cycles, matt areas could be found on the surfaces of the capsules produced. The injection moulding cycle was interrupted, and the injection mould was inspected. Deposit could be seen on the high-gloss polished surfaces of the mould inserts. The deposit was wiped off by means of an acetone-saturated wipe, and analysed. The presence of glycerol monostearate could be detected.

### Example 3

### Comparative Example

As described in Example 1, a mixture (composition) was prepared in the twin-screw extruder, but the vent at the end of the extruder had been sealed.

The moisture content of the resultant pellets was determined by the Karl Fischer method as 1.2% of water.

As described in Example 1, the resultant pellets were charged to the injection moulding machine and processed. The capsules obtained had surface defects, such as streaks, grooves and uneven areas, and did not meet the requirements.

1. Process for producing mouldings by injection moulding, the steps in the process being
A) Melting a mixture made from
a) a (meth)acrylate copolymer composed of from 40 to 100% by weight of free-radical-polymerized C₁-C₄-alkyl esters of acrylic or methacrylic acid and from 0 to 60% by weight of (meth)acrylate monomers having an anionic group in the alkyl radical, where the copolymer comprises
b) from 0.1 to 3% by weight of a release agent, and, where appropriate, the mixture may comprise
c) from 0 to 50% by weight of a drier,
d) from 0 to 30% by weight of a plasticizer,
e) from 0 to 100% by weight of additives or auxiliaries,
f) from 0 to 100% by weight of an active pharmaceutical ingredient,
g) from 0 to 20% by weight of another polymer or copolymer,
where the amounts given for components b) to g) are based on the (meth)acrylate copolymer a) and
the mixture prior to melting has a content of more than 0.5% by weight of low-boiling constituents with vapour pressure of at least 1.9 bar at 120° C.,
B) Devolatilizing the mixture In the thermoplastic state at temperatures of at least 120° C., thereby lowering to not more than 0.5% by weight the content of the low-boiling constituents with vapour pressure of at least 1.9 bar at 120° C.,
C) Injecting the molten and devolatilized mixture into the mould cavity of an injection mould, the temperature of the mould cavity being below the glass transition temperature of the (meth)acrylate copolymer by at least 10° C., cooling the molten mixture, and removing the resultant moulding from the mould.

2. Process according to claim 1, characterized in that the devolatilizing step b) takes place by way of extrusion drying by means of an extruder with devolatilizing section, or by way of an injection moulding plant with a vent in the injection moulding cylinder upstream of the injection mould.

3. Process according to claim 1 or 2, characterized in that the (meth)acrylate copolymer comprises, as (meth)acrylate monomer having an anionic group in the alkyl radical, from 1 to 50% by weight of methacrylic acid.

4. Process according to one or more of claims 1 to 3, characterized in that the mixture comprises from 0.5 to 25% by weight of plasticizer.

5. Injection moulding which can be produced by a process according to one or more of claims 1 to 4.

6. Moulding according to claim 5, characterized in that its impact strength to ISO 179 is at least 5 kJ/m².

7. Moulding according to claim 5 or 6, characterized in that it Is a capsule part of a capsule, or part of a dosage unit.

8. Moulding according to claim 5 or 6, characterized in that it comprises an active pharmaceutical ingredient.

9. Use of a moulding according to one or more of claims 5 to 8 to contain or carry an active pharmaceutical ingredient.
